(19) United States
(12) Patent Application Publication
(10) Pub. No.: US 2016/0296707 A1
(43) Pub. Date: Oct. 13, 2016

(54) SUBRETINAL INJECTION APPARATUS

(71) Applicant: Susan M. Malinowski, Birmingham, MI (US)

(72) Inventor: Susan M. Malinowski, Birmingham, MI (US)

(21) Appl. No.: 15/091,924

(22) Filed: Apr. 6, 2016

Related U.S. Application Data
(60) Provisional application No. 62/144,034, filed on Apr. 7, 2015.

(51) Int. Cl. A61M 5/315; A61M 5/32; A61F 9/00

(52) U.S. Cl. CPC A61M 5/3158; A61F 9/0008; A61M 5/32

(57) ABSTRACT

A subretinal injection device having an injection syringe with a needle adapted for injection behind the retina of an eye. The syringe includes a barrel defining a chamber, an injection needle fluidly connected to the chamber, and a plunger having an inner end and an outer end. The internal end forms an end to the chamber in the syringe. An air pump is fluidly connected to the outer end of the plunger so that air pressure controls the actuation of the syringe.

### CROSS-REFERENCE TO RELATED APPLICATIONS

This application claims priority of U.S. Provisional Application No. 62/144,034 filed Apr. 7, 2015, the contents of which are incorporated herein by reference.

### BACKGROUND OF THE INVENTION

I. Field of the Invention

The present invention relates generally to medical devices and, more particularly, to a subretinal injection device for the injection of fluid under the retina during retinal surgery.

II. Description of Related Art

During retinal surgery it is oftentimes necessary to inject fluids underneath the retina. Injection of fluids underneath the retina is difficult to accomplish for a number of different reasons.

For example, the amount of space underneath the retina is very limited. As such, in order to inject the fluid underneath the retina it is necessary to insert a cannula into the eyeball so that the open end of the cannula is positioned beneath the retina. This is typically accomplished by using a conventional syringe.

However, in order to actually inject the fluid underneath the retina, it is necessary for the surgeon to depress the syringe actuator in order to inject the fluid from the cannula and simultaneously maintain the open end of the cannula essentially motionless so that it remains underneath the retina. This procedure is difficult to perform especially for surgeons with small hands. Furthermore, in the event that the syringe moves during the injection process, unacceptable damage to the retina can result.

### SUMMARY OF THE PRESENT INVENTION

The present invention provides a medical device which overcomes the above-mentioned disadvantages of the previously known retinal injection devices.

In brief, the present invention provides a subretinal injection device having an injection syringe. The injection syringe includes a needle adapted for insertion behind the retina of the eye. The syringe includes a barrel which defines a chamber which contains the fluid for injection.

An injection needle is then fluidly connected to one end of the syringe chamber. The other end of the syringe chamber is closed by a plunger which is slidably mounted to the syringe. Consequently, upon depression of the plunger into the syringe, the plunger forces the fluid contained within the syringe out through the needle.

In order to control the actuation of the plunger, and thus the injection of fluid into the eye, an air pump is fluidly connected to the outer end of the plunger. Consequently, upon an increase of the air pressure by the air pump, the plunger is displaced within the syringe thus injecting the fluid from the syringe.

Preferably, the air pump includes an injection needle which is fluidly connected to the plunger by a flexible tube. The tube, however, is fluidly sealed. Consequently, fluid can be displaced from the injection syringe by simply increasing the air pressure to the injection syringe but without manually moving the injection syringe.

### BRIEF DESCRIPTION OF THE DRAWING

A better understanding of the present invention will be had upon reference to the following detailed description when read in conjunction with the accompany drawing, wherein like reference characters refer to like parts throughout the several views, and in which:

FIG. 1 is a view illustrating a preferred embodiment of the subretinal injection device of the present invention.

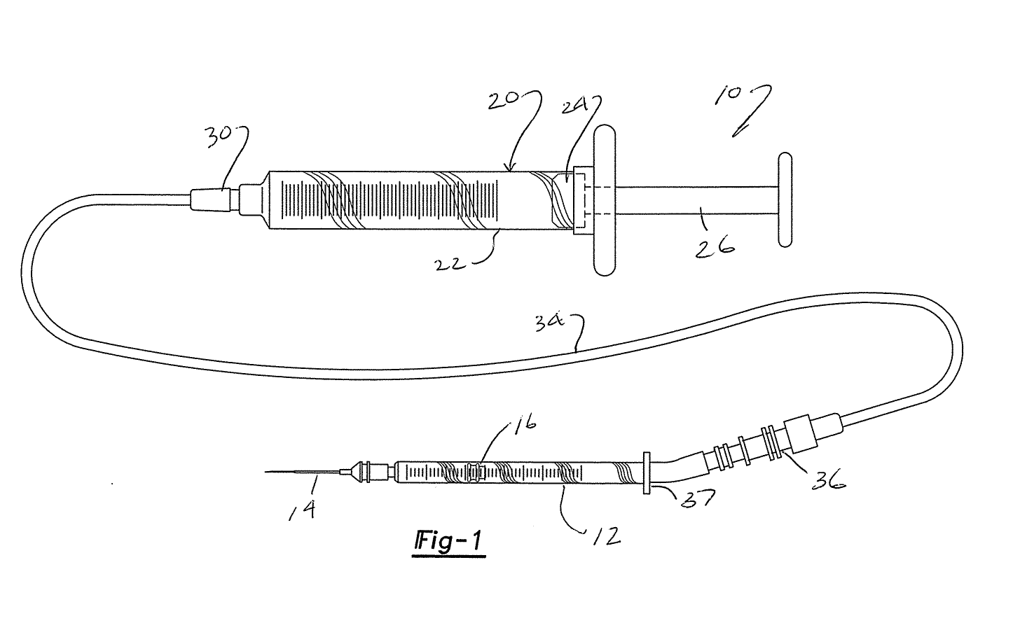

### DETAILED DESCRIPTION OF A PREFERRED EMBODIMENT OF THE PRESENT INVENTION

With reference to the drawing, a preferred embodiment of an apparatus 10 according to the present invention is shown for subretinal injection. The device 10 includes a first syringe 12 with an outwardly extending cannula or needle 14. The needle 14 is adapted for insertion into the patient's eye behind the retina.

In the conventional fashion, the syringe 12 includes a plunger 16 within the syringe 12 so that the displacement of the plunger 16 injects fluid from the open end of the cannula 14. Consequently, the entire volume between the plunger 16 and the cannula 14 is filled with a fluid intended for injection during the medical procedure.

In order to axially displace the plunger 16 in the syringe 12, the syringe 12 is fluidly connected by a tube 34 to an air pump 20. Although any conventional means may be used to fluidly connect the syringe 12 to the air pump 20, the fluid connection between the cylinder 20 and the syringe 12 is airtight and includes an elongated flexible conduit 34.

The air pump 20 preferably includes a plunger 34 slidably mounted within an elongated tube 22. The plunger 24 is initially positioned adjacent one end of the tube 22 while the elongated flexible tube 34 is fluidly connected to the opposite end 30 of the cylinder 20. Consequently, displacement of the plunger 24 within the cylinder 20 fluidly pumps air from the cylinder 20, through the tube 24, and into the syringe 12 at the end of the plunger 16 opposite from the injection cannula 14.

Any means may be used to displace the plunger 24 in the tube 22 in order to inject fluid from the syringe 12 out through the cannula 14. However, as shown in the drawing, an actuator handle 26 is preferably connected to the plunger 24 so that the plunger 24 may be manually displaced in its tube 22 by the doctor or other medical personnel. Furthermore, a handle 26 is preferably connected to the plunger 24 to facilitate the manual displacement of the plunger 24 in the fluid pump 22 and thus the displacement of the plunger 16 in the syringe 12.

During an operation, the cannula 14 is first inserted by the doctor behind the retina. Thereafter, injection of fluid from the syringe 12 is controlled by displacing the actuator 26. Since the actuator 26 is only indirectly fluidly connected to the syringe 12 by the flexible tube 34, the syringe 12 remains stationary during the injection process.

Having described my invention, many modifications thereto will become apparent to those skilled in the art to which it pertains without deviation from the spirit of the invention as defined by the scope of the appended claims.

I claim:

1. A subretinal injection device comprising:
an injection syringe having a needle adapted for injection behind a retina of an eye, said syringe having a barrel defining a chamber, an injection needle fluidly connected to said chamber and a plunger having an inner end and an outer end, said inner end forming an end of said chamber opposite from said needle, and
an air pump fluidly connected to an outer end of said plunger.

2. The device as defined in claim 1 wherein said air pump comprises an actuation syringe.

3. The device as defined in claim 2 wherein said actuation syringe is fluidly connected to said outer end of said plunger.

4. The device as defined in claim 2 wherein said actuation syringe is fluidly connected to said outer end of said plunger by a flexible tube.

5. The device as defined in claim 4 wherein said flexible tube is gas filled.

6. The device as defined in claim 5 wherein said flexible tube is air filled.

7. The device as defined in claim 6 wherein said flexible tube is sealed.
